(19) United States
(12) Patent Application Publication
(10) Pub. No.: US 2026/0001775 A1
(43) Pub. Date: Jan. 1, 2026

(54) PREPARATION METHOD OF MOLYBDENITE/ZNO NANOROD-BASED PILLARED STRUCTURE AND APPLICATION THEREOF

(71) Applicant: Kunming University of Science and Technology, Kunming (CN)

(72) Inventors: Yi Xia, Kunming (CN); Qingfu Dai, Kunming (CN); Shenghui Guo, Kunming (CN); Li Yang, Kunming (CN); Hong Li, Kunming (CN)

(21) Appl. No.: 19/331,961

(22) Filed: Sep. 17, 2025

(30) Foreign Application Priority Data
Sep. 20, 2024 (CN) 2024113117854

(51) Int. Cl. C01G 39/06; C01G 9/02; G01N 33/00

(52) U.S. Cl. CPC C01G 39/06; C01G 9/02; G01N 33/0037; C01P 2004/16

(57) ABSTRACT

A preparation method for a molybdenite/ZnO nanorod-based pillared structure and an application thereof are provided, belonging to the field of gas sensing technology. The specific steps include: (1) dispersing commercially available molybdenite in a zinc acetate methanol solution to obtain a solution A; (2) adding a sodium hydroxide methanol solution to the solution A to obtain molybdenite/ZnO nanoparticles; (3) dispersing the molybdenite/ZnO nanoparticles in deionized water to obtain a molybdenite/ZnO nanoparticle suspension; (4) adding the molybdenite/ZnO nanoparticle suspension to a sodium hydroxide solution of ε-Zn(OH)₂, heating and aging, stirring, washing, and drying overnight to obtain molybdenite/ZnO nanorods with pillared heterostructure.

### CROSS REFERENCE TO RELATED APPLICATIONS

The present disclosure claims the priority to the Chinese patent application with the filing No. 202411311785.4, entitled “PREPARATION METHOD OF MOLYBDENITE/ZNO NANOROD-BASED PILLARED STRUCTURE AND APPLICATION THEREOF” and filed on Sep. 20, 2024 with the Chinese Patent Office, the contents of which are incorporated herein by reference in their entirety.

### TECHNICAL FIELD

The present disclosure relates to the field of gas sensing technology, and more particularly to a preparation method of a molybdenite/ZnO nanorod-based pillared structure and the application thereof in NO₂ monitoring.

### BACKGROUND ART

Nitrogen dioxide (NO₂) is one of the most toxic air pollutants, mainly derived from the combustion of fossil fuels and industrial production. In addition to causing acid rain and photochemical smog and damaging the ozone layer, it also has a significant impact on human health. For example, respiratory diseases: it irritates the respiratory tract, triggers or exacerbates diseases such as asthma and bronchitis, and increases the risk of developing cardiovascular diseases. Impairment of the immune system: it reduces immune function and increases the risk of infection. Even at the ppb level, nitrogen dioxide (NO₂) is severely harmful. The U.S. Environmental Protection Agency (EPA) has set the air quality control standard for NO₂ at 53 ppb. Continuous or frequent exposure to NO₂ with the concentration exceeding the air quality standard 53 ppb may increase the morbidity of acute respiratory and olfactory paralysis. Therefore, it is of great significance to realize convenient and timely monitoring of trace NO₂.

Nitrogen dioxide (NO₂) detection is achieved through its adsorption and activation at the adsorption and active sites on the surface of the sensitive material, and change in the electrical resistivity of the sensitive material caused by occurrence of transferring of the charges of the sensitive material after activation. However, materials commonly used for NO₂ detection are primarily metal oxide-based semiconductors, which have been extensively studied due to their simple preparation methods and low cost. ZnO, in particular, has emerged as one of the most promising NO₂ sensing materials due to its high sensitivity and high selectivity for NO₂. However, metal oxides typically require relatively high operating temperatures (150-400° C.), limiting their application at low temperatures. UV activation is considered an effective strategy for achieving NO₂ sensing at room temperature via various semiconductor nanostructures; however, this strategy still requires relatively harsh ultraviolet light irradiation.

It is found through researches that MoS₂, the primary component of molybdenite, possesses high specific surface area, abundant active sites, excellent electrical properties, and easy integration with other materials. It has recently been widely used in the field of lubricants, catalysts, photocatalysts, electronic devices, energy storage devices, and gas sensors. MoS₂ exhibits high sensitivity, good selectivity, and low operating temperature for NO₂ detection, and MoS₂ has a multilayered structure that facilitates NO₂ adsorption and transport. However, current methods for preparing MoS₂ primarily rely on mechanical exfoliation, liquid-phase exfoliation, and hydrothermal/solvothermal methods. These preparation methods require high energy consumption, complex equipment, and low MoS₂ yields. The present disclosure, however, directly utilizes commercially available molybdenite as a raw material to prepare gas-sensitive materials, reducing material preparation costs. However, the stacking and aggregation of molybdenite (MoS₂) result in an insufficient gas permeability and a lack of surface active sites involved in the sensing process. Therefore, the construction of pillared structure is considered a promising strategy to address the stacking and agglomeration issues of two-dimensional materials, which may be achieved by inserting and growing specific nanostructures between layers. The pillared structure has the advantage of a larger surface area, increasing the exposure of active sites and enhancing the transfer of charge carriers.

Therefore, providing a pillared molybdenite/ZnO gas sensing material that may respond to ppb-level NO₂ and a preparation method thereof is an urgent problem to be solved by those skilled in the art.

### SUMMARY

In view of this, the present disclosure provides a method for preparing a molybdenite/ZnO nanorod-based pillared structure and the application thereof. The method has a simple preparation process that does not involve the use of advanced equipment, facilitating industrial production of the material. The molybdenite/ZnO pillared heterostructure material synthesized by the method exhibits a response to ppb-level NO₂ under simulated sunlight irradiation at a room temperature.

One of the purposes of the present disclosure is to provide a method for preparing a molybdenite/ZnO nanorod-based pillared structure, including the following steps:
(1) dispersing commercially available molybdenite into a zinc acetate methanol solution under stirring until adsorption equilibrium is reached, to obtain a solution A for later use;
(2) adding sodium hydroxide methanol solution to the solution A, stirring continuously, centrifuging, washing and drying to obtain molybdenite/ZnO nanoparticles;
(3) dispersing the molybdenite/ZnO nanoparticles in deionized water and stirring evenly to obtain a molybdenite/ZnO nanoparticle suspension for later use; and
(4) adding the molybdenite/ZnO nanoparticle suspension to a sodium hydroxide solution of ε-Zn(OH)₂ under stirring, stirring for 10 min, heating for aging, stirring again, washing, and drying overnight to obtain the molybdenite/ZnO nanorods with a pillared heterostructure.

Further, the purity of MoS₂ in the commercially available molybdenite is ≥99%.

Further, in step (1), the solid-liquid ratio of the commercially available molybdenite to the zinc acetate methanol solution is 1 mg: 1 mL; and the concentration of the zinc acetate methanol solution is 2.0 mol/L.

Further, in step (2), the concentration of the sodium hydroxide methanol solution is 2.0-16.0 mol/L; the molar ratio of zinc ion to sodium ion in step (2) is 1:6.25-25; the stirring rate is 0-600 rpm, the centrifugal speed is 2000 rpm, and the drying is performed at 20-30° C. for 12-24 h.

The beneficial effect of adopting the above technical solution was that under this process, the zinc oxide nanoparticles form a better pillared structure between the layers of molybdenite. The low-speed centrifugation is to avoid destroying the pillared structure.

Further, in step (3), the solid-liquid ratio of the molybdenite/ZnO nanoparticles to deionized water is 10 mg: 1 mL; and the stirring rate is 0-600 rpm.

Further, in step (4), the preparation method of the ε-Zn(OH)₂ is as follows: dropwise adding sodium hydroxide solution to zinc sulfate solution under stirring, for sufficient reaction, washing the precipitate and drying to obtain ε-Zn(OH)₂, where the stirring rate is 0-600 rpm; the aging temperature is 60-100° C., and the aging time is 0.5 h.

Further, the concentration of the sodium hydroxide solution is 2.0-4.0 mol/L, the concentration of the zinc sulfate solution is 1.0-2.0 mol/L, and the molar ratio of the sodium hydroxide to the zinc sulfate is 2:1; the stirring rate is 100-700 rpm, and the drying is carried out at 15-20° C. for 22-26 h.

Further, in step (4), the mass ratio of the ε-Zn(OH)₂ to the molybdenite/ZnO nanoparticles is 3.48:0.1, the liquid-solid ratio of the sodium hydroxide solution to the molybdenite/ZnO nanoparticles is 200 mL: 0.1 g, and the concentration of the sodium hydroxide solution is 4.0 mol/L; the stirring rate is 200-800 rpm, and the stirring is performed again for 20-40 min, and the drying condition is 60° C.

The beneficial effects of the above technical solutions are as follows: under this process condition, the molybdenite/ZnO nanorod-based pillared structure prepared by using ε-Zn(OH)₂ as a precursor and molybdenite/ZnO nanoparticles as crystal seed is more stable, and has higher sensing sensitivity and lower detection limit. In addition, the zinc oxide nanoparticles grown between the molybdenite layers increase electron transport channels, adsorption sites, and active sites, thereby improving stability and sensitivity. The formation of zinc oxide nanorods on the molybdenite/ZnO surface increase more adsorption sites and active sites, resulting in better sensing performance.

Further preferably, the purity of zinc acetate dihydrate used to prepare the zinc acetate methanol solution is ≥99%, and the purity of methanol is ≥99.5%; and the purity of the sodium hydroxide is ≥99.5%, and the purity of the zinc sulfate is ≥99%.

The technical concept of the present disclosure is as follows: MoS₂ has a high specific surface area and abundant active sites. MoS₂ has a high sensitivity, good selectivity and low operating temperature for NO₂ detection, and MoS₂ has a multilayered structure, which is conducive to the adsorption and transmission of NO₂. However, due to the stacking of the layered structure, the active sites are reduced, reducing the gas-sensitive performance, and the pillared structure may well solve the problem of MoS₂ stacking. Moreover, Zinc oxide nanorods exhibit characteristics such as a high specific surface area, excellent electronic properties, diverse morphologies, controllable sizes, good chemical stability and thermal stability, low cost, and easy preparation. Therefore, pillared MoS₂ is combined with zinc oxide nanorods to fabricate high-performance gas-sensitive materials.

The active sites of the molybdenite/ZnO nanorod composite material in the present disclosure are oxygen active sites. After NO₂ is adsorbed by the composite material, it is oxidized by these oxygen active sites, resulting in a change in the electrical resistivity of the material. The formation mechanism is that the molybdenite/ZnO nanoparticles and zinc oxide nanorods form a heterostructure, while modifying the morphology of zinc oxide on the surface of molybdenite/ZnO nanoparticles may also increase their specific surface areas and active sites.

The second purpose of the present disclosure is to provide an application of a molybdenite/ZnO nanorod-based pillared structure, where the molybdenite/ZnO nanorod-based pillared structure is used to monitor 50 ppb NO₂ under a simulated sunlight condition at a room temperature.

Because molybdenite with a layered structure has good adsorption capacity and abundant active sites, NO₂ is easily oxidized on the surface of the material, while the pillared structure improves the stability of the material and enhances its electron transport ability and sensitivity, and the zinc oxide nanorods are grown on the surface of molybdenite/ZnO nanoparticles, increasing the active sites and enhancing the sensitivity and low-temperature activity of the material.

The key to forming the pillared molybdenite/ZnO nanoparticles of the present disclosure lies in the stirring rate, the concentration of the zinc acetate methanol solution and the concentration of the sodium hydroxide methanol solution, as well as the dropping speed of the zinc acetate methanol solution. The formation of pillared molybdenite/ZnO nanorods is jointly determined by the stirring rate, the concentration of the sodium hydroxide solution, the amount of molybdenite/ZnO seed crystals used, the amount of the ε-Zn(OH)₂ precursor used, the aging time, and the aging temperature.

It may be seen from the above technical solutions that compared with the prior art, the beneficial effects of the present disclosure are as follows.
(1) The gas-sensitive material of the present disclosure has the characteristics of simple preparation method, low cost, etc. The molybdenite/ZnO nanorods prepared by the above method have good NO₂ response performance.
(2) The method employs an adjustable molar ratio of zinc ions to sodium ions, which allows for the regulation of its NO₂ adsorption and transport capabilities, and simultaneously, and regulates the number of active sites and electron transport capability, highly valuable in the field of room-temperature NO₂ sensing.

This method has a low synthesis temperature. Except for the high aging temperature, the rest of the synthesis process is completed at room temperature. Compared with other synthesis methods, it is more environmentally friendly.

### BRIEF DESCRIPTION OF DRAWINGS

In order to more clearly illustrate the technical solutions of the embodiments of the present disclosure or in the prior art, the drawings required for use in description of the embodiments or the prior art will be briefly introduced below. Obviously, the drawings described below are merely embodiments of the present disclosure. For a person ordinarily skilled in the art, other drawings may be obtained based on the provided drawings without paying any creative work.

FIG. 1 is a SEM image of molybdenite/ZnO nanorods prepared in Example 3.

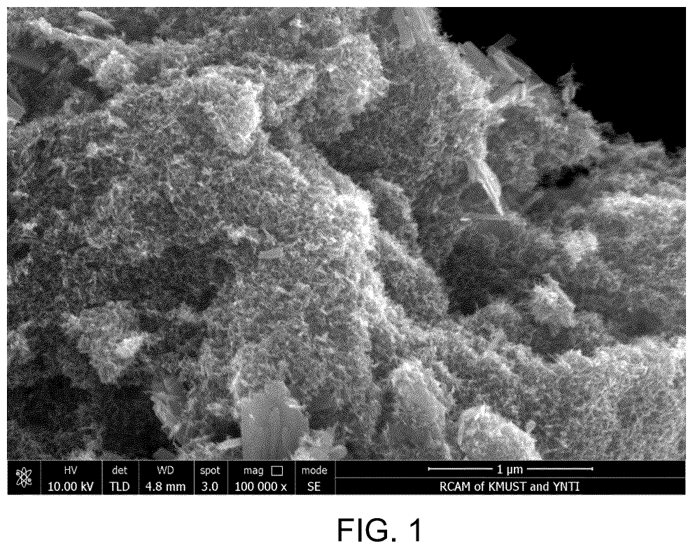

FIG. 2 is a graph showing the response of the molybdenite/ZnO nanorods prepared in Example 3 to 50 ppb NO₂.

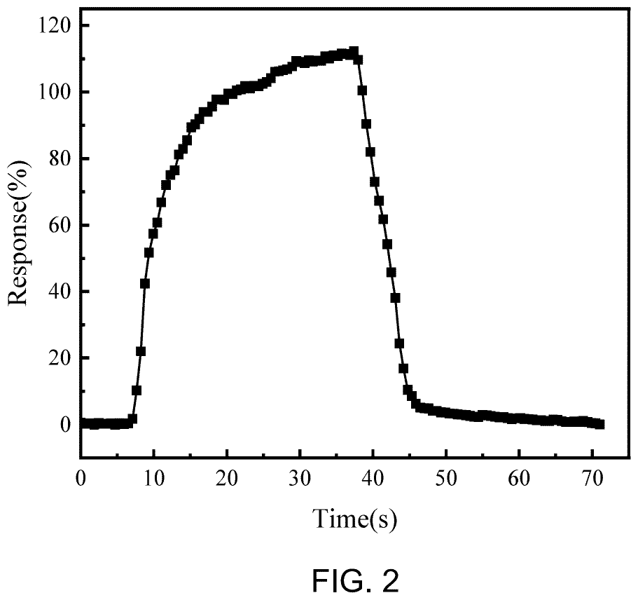

### DETAILED DESCRIPTION OF EMBODIMENTS

The technical solutions in the embodiments of the present disclosure will be clearly and completely described below in conjunction with the drawings in the embodiments of the present disclosure. Obviously, the described embodiments are only some of the embodiments of the present disclosure, not all of the embodiments. Based on the embodiments of the present disclosure, all other embodiments obtained by a person ordinarily skilled in the art without making creative efforts are within the scope of protection of the present disclosure.

In the examples of the present disclosure, the purity of MoS₂ in molybdenite is ≥99%, the purity of zinc acetate dihydrate is ≥99%, the purity of methanol is ≥99.5%, the purity of sodium hydroxide is ≥99.5%, and the purity of zinc sulfate is ≥99%.

### Example 1

A preparation method of gas-sensitive material with molybdenite/ZnO pillared heterostructure:
(1) dispersing 100 mg of molybdenum ore in 100 ml of a methanol solution of zinc acetate dihydrate with a concentration of 2.0 mol/L, stirring at a room temperature for 1 h until the adsorption equilibrium was reached, adding 312.5 mL of a methanol solution of sodium hydroxide with a concentration of 4.0 mol/L, where the molar ratio of zinc ions to sodium hydroxide was 1:6.25, stirring at 300 rpm for 2 h, then centrifuging at 2000 rpm for 5 min, washing with deionized water for 5 times, followed by washing with ethanol for 2 times, and vacuum-drying at 25° C. for 24 h to obtain pillared molybdenite/ZnO nanoparticles;
(2) dropping 100 mL of 2.0 mol/L zinc sulfate solution into 100 mL of 4.0 mol/L sodium hydroxide solution at 300 rpm, stirring for 2 h, filtering and then drying at 18° C. for 24 h; and
(3) dispersing 100 mg of molybdenite/ZnO nanoparticles in 10 mL of deionized water, adding to 200 mL of 4.0 mol/L sodium hydroxide solution containing 3.48 g of dissolved ε-Zn(OH)₂ under stirring at 300 rpm, stirring for 10 min, heating to 80° C., aging for 0.5 h, then stirring at 300 rpm, washing, and drying at 60° C. overnight to obtain molybdenite/ZnO nanorods.

In the gas-sensing nanomaterial of pillared molybdenite/ZnO nanorods synthesized in Example 1, the molar ratio of zinc ions to sodium hydroxide was 1:6.25. The material exhibited a response to a NO₂ concentration of 50 ppb under simulated sunlight irradiation. It responded 35 s after NO₂ was introduced into the gas chamber, and when NO₂ and other reactants were removed from the gas chamber, the resistance of the pillared molybdenite/ZnO nanorod pillared heterojunction material may return to 50% of its initial state, indicating a general stability.

### Example 2

A preparation method of gas-sensitive material with molybdenite/ZnO pillared heterostructure:
(1) dispersing 100 mg of molybdenite in 100 ml of a methanol solution of zinc acetate dihydrate with a concentration of 2.0 mol/L, stirring at a room temperature for 1 h until the adsorption equilibrium was reached, adding 312.5 mL of a methanol solution of sodium hydroxide with a concentration of 8.0 mol/L, where the molar ratio of zinc ions to sodium hydroxide was 1:12.5, stirring at 300 rpm for 2 h, then centrifuging at 2000 rpm for 5 min, washing with deionized water for 5 times, followed by washing with ethanol for 2 times, and vacuum-drying at 25° C. for 24 h to obtain pillared molybdenite/ZnO nanoparticles;
(2) dropping 100 mL of 2.0 mol/L zinc sulfate solution into 100 mL of 4.0 mol/L sodium hydroxide solution at 300 rpm, stirring for 2 h, filtering and then drying at 18° C. for 24 h; and
(3) dispersing 100 mg of molybdenite/ZnO nanoparticles in 10 mL of deionized water, adding to 200 mL of 4.0 mol/L sodium hydroxide solution containing 3.48 g of dissolved ε-Zn(OH)₂ under stirring at 300 rpm, stirring for 10 min, heating to 80° C., aging for 0.5 h, then stirring at 300 rpm, washing, and drying at 60° C. overnight to obtain molybdenite/ZnO nanorods.

In the gas-sensing nanomaterial of pillared molybdenite/ZnO nanorods synthesized in Example 2, the molar ratio of zinc ions to sodium hydroxide was 1:12.5. The material exhibited a response to a NO₂ concentration of 50 ppb under simulated sunlight irradiation. It responded 19 s after NO₂ was introduced into the gas chamber, and when NO₂ and other reactants were removed from the gas chamber, the resistance of the pillared molybdenite/ZnO nanorod pillared heterojunction material may return to 76% of its initial state, indicating a general stability.

### Example 3

A preparation method of gas-sensitive material with molybdenite/ZnO pillared heterostructure:
(1) dispersing 100 mg of molybdenite in 100 ml of a methanol solution of zinc acetate dihydrate with a concentration of 2.0 mol/L, stirring at a room temperature for 1 h until the adsorption equilibrium was reached, adding 625 mL of a methanol solution of sodium hydroxide with a concentration of 8.0 mol/L, where the molar ratio of zinc ions to sodium hydroxide was 1:25, stirring at 300 rpm for 2 h, then centrifuging at 2000 rpm for 5 min, washing with deionized water for 5 times, followed by washing with ethanol for 2 times, and vacuum-drying at 25° C. for 24 h to obtain pillared molybdenite/ZnO nanoparticles;
(2) dropping 100 ml of 2.0 mol/L zinc sulfate solution into 100 mL of 4.0 mol/L sodium hydroxide solution at 300 rpm, stirring for 2 h, filtering and then drying at 18° C. for 24 h; and
(3) dispersing 100 mg of molybdenite/ZnO nanoparticles in 10 mL of deionized water, adding to 200 mL of 4.0 mol/L sodium hydroxide solution containing 3.48 g of dissolved ε-Zn(OH)₂ under stirring at 300 rpm, stirring for 10 min, heating to 80° C., aging for 0.5 h, then stirring at 300 rpm, washing, and drying at 60° C. overnight to obtain molybdenite/ZnO nanorods.

In the gas-sensing nanomaterial of pillared molybdenite/ZnO nanorods synthesized in Example 3, the molar ratio of zinc ions to sodium hydroxide was 1:25. The material exhibited a response to a NO₂ concentration of 50 ppb. It responded 5 s after NO₂ was introduced into the gas chamber, and when NO₂ and other reactants were removed from the gas chamber, the resistance of the pillared molybdenite/ZnO nanorod pillared heterojunction material may return to 95% of its initial state, indicating an excellent stability.

### Example 4

A preparation method of gas-sensitive material with molybdenite/ZnO pillared heterostructure:
(1) dispersing 100 mg of molybdenite in 100 ml of a methanol solution of zinc acetate dihydrate with a concentration of 2.0 mol/L, stirring at a room temperature for 1 h until the adsorption equilibrium was reached, adding 312.5 mL of a methanol solution of sodium hydroxide with a concentration of 16.0 mol/L, where the molar ratio of zinc ions to sodium hydroxide was 1:25, stirring at 300 rpm for 2 h, then centrifuging at 2000 rpm for 5 min, washing with deionized water for 5 times, followed by washing with ethanol for 2 times, and vacuum-drying at 25° C. for 24 h to obtain pillared molybdenite/ZnO nanoparticles;
(2) dropping 100 mL of 2.0 mol/L zinc sulfate solution into 100 mL of 4.0 mol/L sodium hydroxide solution at 300 rpm, stirring for 2 h, filtering and then drying at 18° C. for 24 h; and
(3) dispersing 100 mg of molybdenite/ZnO nanoparticles in 10 mL of deionized water, adding to 200 mL of 4.0 mol/L sodium hydroxide solution containing 3.48 g of dissolved ε-Zn(OH)₂ under stirring at 300 rpm, stirring for 10 min, heating to 80° C., aging for 0.5 h, then stirring at 300 rpm, washing, and drying at 60° C. overnight to obtain molybdenite/ZnO nanorods.

In the gas-sensing nanomaterial of pillared molybdenite/ZnO nanorods synthesized in Example 4, the molar ratio of zinc ions to sodium hydroxide was 1:25. The material exhibited a response to a NO₂ concentration of 50 ppb under simulated sunlight irradiation. It responded 30 s after NO₂ was introduced into the gas chamber, and when NO₂ and other reactants were removed from the gas chamber, the resistance of the pillared molybdenite/ZnO nanorod pillared heterojunction material may return to 60% of its initial state, indicating a general stability.

### Example 5

A preparation method of gas-sensitive material with molybdenite/ZnO pillared heterostructure:
(1) dispersing 100 mg of hydrothermally synthesized MoS₂ in 100 mL of a methanol solution of zinc acetate dihydrate with a concentration of 2.0 mol/L, stirring at a room temperature for 1 h until the adsorption equilibrium was reached, adding 625 mL of a methanol solution of sodium hydroxide with a concentration of 8.0 mol/L, where the molar ratio of zinc ions to sodium hydroxide was 1:25, stirring at 300 rpm for 2 h, then centrifuging at 2000 rpm for 5 min, washing with deionized water for 5 times, followed by washing with ethanol for 2 times, and vacuum-drying at 25° C. for 24 h to obtain pillared molybdenite/ZnO nanoparticles;
(2) dropping 100 mL of 2.0 mol/L zinc sulfate solution into 100 mL of 4.0 mol/L sodium hydroxide solution at 300 rpm, stirring for 2 h, filtering and then drying at 18° C. for 24 h; and
(3) dispersing 100 mg of molybdenite/ZnO nanoparticles in 10 mL of deionized water, adding to 200 mL of 4.0 mol/L sodium hydroxide solution containing 3.48 g of dissolved ε-Zn(OH)₂ under stirring at 300 rpm, stirring for 10 min, heating to 80° C., aging for 0.5 h, then stirring at 300 rpm, washing, and drying at 60° C. overnight to obtain molybdenite/ZnO nanorods.

In the gas-sensing nanomaterial of pillared molybdenite/ZnO nanorods synthesized in Example 5, the molar ratio of zinc ions to sodium hydroxide was 1:25. The material exhibited a response to a NO₂ concentration of 50 ppb under simulated sunlight irradiation. It responded 5 s after NO₂ was introduced into the gas chamber, and when NO₂ and other reactants were removed from the gas chamber, the resistance of the pillared molybdenite/ZnO nanorod pillared heterojunction material may return to 96% of its initial state, indicating an excellent stability.

### Example 6

A preparation method of gas-sensitive material with molybdenite/ZnO pillared heterostructure:
(1) dispersing 100 mg of molybdenite, in which the purity of MoS₂ is 90%, in 100 ml of a methanol solution of zinc acetate dihydrate with a concentration of 2.0 mol/L, stirring at a room temperature for 1 h until the adsorption equilibrium was reached, adding 625 mL of a methanol solution of sodium hydroxide with a concentration of 8.0 mol/L, where the molar ratio of zinc ions to sodium hydroxide was 1:25, stirring at 300 rpm for 2 h, then centrifuging at 2000 rpm for 5 min, washing with deionized water for 5 times, followed by washing with ethanol for 2 times, and vacuum-drying at 25° C. for 24 h to obtain pillared molybdenite/ZnO nanoparticles;
(2) dropping 100 mL of 2.0 mol/L zinc sulfate solution into 100 mL of 4.0 mol/L sodium hydroxide solution at 300 rpm, stirring for 2 h, filtering and then drying at 18° C. for 24 h; and
(3) dispersing 100 mg of molybdenite/ZnO nanoparticles in 10 mL of deionized water, adding to 200 mL of 4.0 mol/L sodium hydroxide solution containing 3.48 g of dissolved ε-Zn(OH)₂ under stirring at 300 rpm, stirring for 10 min, heating to 80° C., aging for 0.5 h, then stirring at 300 rpm, washing, and drying at 60° C. overnight to obtain molybdenite/ZnO nanorods.

In the gas-sensing nanomaterial of pillared molybdenite/ZnO nanorods synthesized in Example 6, the molar ratio of zinc ions to sodium hydroxide was 1:20. The material exhibited a response to a NO₂ concentration of 50 ppb under simulated sunlight irradiation. It responded 13 s after NO₂ was introduced into the gas chamber, and when NO₂ and other reactants were removed from the gas chamber, the resistance of the pillared molybdenite/ZnO nanorod pillared heterojunction material may return to 86% of its initial state, indicating a good stability.

### Example 7

A preparation method of gas-sensitive material with molybdenite/ZnO pillared heterostructure:
(1) dispersing 100 mg of molybdenite in 100 ml of a methanol solution of zinc acetate dihydrate with a concentration of 2.0 mol/L, stirring at a room temperature for 1 h until the adsorption equilibrium was reached, adding 625 mL of a methanol solution of sodium hydroxide with a concentration of 8.0 mol/L, where the molar ratio of zinc ions to sodium hydroxide was 1:25, stirring at 300 rpm for 2 h, then centrifuging at 2000 rpm for 5 min, washing with deionized water for 5 times, followed by washing with ethanol for 2 times, and vacuum-drying at 25° C. for 24 h to obtain pillared molybdenite/ZnO nanoparticles;
(2) dropping 100 ml of 2.0 mol/L zinc sulfate solution into 100 ml of 4.0 mol/L sodium hydroxide solution at 300 rpm, stirring for 2 h, filtering and then drying at 18° C. for 24 h; and
(3) dispersing 100 mg of molybdenite/ZnO nanoparticles in 10 mL of deionized water, adding to 200 mL of 4.0 mol/L sodium hydroxide solution containing 3.48 g of dissolved ε-Zn(OH)₂ under stirring at 300 rpm, stirring for 10 min, aging for 0.5 h at 60° C. and 100° C. respectively, then stirring at 300 rpm, washing, and drying at 60° C. overnight to obtain molybdenite/ZnO nanorods.

In the gas-sensing nanomaterial of pillared molybdenite/ZnO nanorods synthesized in Example 7, the molar ratio of zinc ions to sodium hydroxide was 1:25. The material exhibited a response to a NO₂ concentration of 50 ppb under simulated sunlight irradiation. It responded 20 s and 25 s respectively after NO₂ was introduced into the gas chamber, and when NO₂ and other reactants were removed from the gas chamber, the resistance of the pillared molybdenite/ZnO nanorod pillared heterojunction material may return to 80% and 78% of its initial state, indicating a good stability.

The above description of the disclosed embodiments is intended to enable one skilled in the art to implement or use the present disclosure. Various modifications to these embodiments will be apparent to one skilled in the art, and the general principles defined herein may be implemented in other embodiments without departing from the spirit or scope of the present disclosure. Therefore, the present disclosure is not limited to the embodiments shown herein but is intended to conform to the widest scope consistent with the principles and novel features disclosed herein.

1. A method for preparing a molybdenite/ZnO nanorod-based pillared structure, comprising following steps:
(1) dispersing molybdenite into a zinc acetate methanol solution under stirring until adsorption equilibrium is reached, to obtain a solution A for later use;
(2) adding a sodium hydroxide methanol solution to the solution A, stirring continuously, centrifuging, washing and drying to obtain molybdenite/ZnO nanoparticles;
(3) dispersing the molybdenite/ZnO nanoparticles in deionized water and stirring evenly to obtain a molybdenite/ZnO nanoparticle suspension for later use; and
(4) adding the molybdenite/ZnO nanoparticle suspension to a sodium hydroxide solution of ε-Zn(OH)₂ under stirring, stirring for 10 min, heating for aging, stirring again, washing and drying overnight to obtain molybdenite/ZnO nanorods with a pillared heterostructure.

2. The method for preparing a molybdenite/ZnO nanorod-based pillared structure according to claim 1, wherein a purity of MoS₂ in the molybdenite is ≥99%.

3. The method for preparing a molybdenite/ZnO nanorod-based pillared structure according to claim 1, wherein in step (1), a solid-liquid ratio of the molybdenite to the zinc acetate methanol solution is 1 mg: 1 mL; and
a concentration of the zinc acetate methanol solution is 2.0 mol/L.

4. The method for preparing a molybdenite/ZnO nanorod-based pillared structure according to claim 1, wherein in step (2), a concentration of the sodium hydroxide methanol solution is 2.0-16.0 mol/L;
in step (2), a molar ratio of zinc ions to sodium ions is 1:6.25-25; and
a stirring rate is 0-600 rpm, a centrifugal speed is 2000 rpm, and the drying is performed at a temperature of 20-30° C. for 12-24 hours.

5. The method for preparing a molybdenite/ZnO nanorod-based pillared structure according to claim 1, wherein in step (3), a solid-liquid ratio of the molybdenite/ZnO nanoparticles to the deionized water is 10 mg: 1 mL; and a stirring rate is 0-600 rpm.

6. The method for preparing a molybdenite/ZnO nanorod-based pillared structure according to claim 1, wherein in step (4), a preparation method of the ε-Zn(OH)₂ comprises: adding a sodium hydroxide solution dropwise to a zinc sulfate solution under stirring, for sufficient reaction, washing and drying a precipitate to obtain ε-Zn(OH)₂; and
a stirring rate is 0-600 rpm; an aging temperature is 60-100° C., and an aging time is 0.5 h.

7. The method for preparing a molybdenite/ZnO nanorod-based pillared structure according to claim 6, wherein a concentration of the sodium hydroxide solution is 2.0-4.0 mol/L, a concentration of the zinc sulfate solution is 1.0-2.0 mol/L, and a molar ratio of sodium hydroxide to zinc sulfate is 2:1; and
a stirring rate is 100-700 rpm, and the drying is carried out at 15-20° C. for 22-26 h.

8. The method for preparing a molybdenite/ZnO nanorod-based pillared structure according to claim 1, wherein in step (4), a mass ratio of the ε-Zn(OH)₂ to the molybdenite/ZnO nanoparticles is 3.48:0.1, a liquid-solid ratio of the sodium hydroxide solution to the molybdenite/ZnO nanoparticles is 200 mL: 0.1 g, and a concentration of the sodium hydroxide solution is 4.0 mol/L; and
a stirring rate is 200-800 rpm, the stirring is performed again for 20-40 min, and a drying condition is 60° C.

9. The method for preparing a molybdenite/ZnO nanorod-based pillared structure according to any one of claims 1 to 8, wherein a purity of zinc acetate dihydrate used to prepare the zinc acetate methanol solution is ≥99%, and a purity of methanol is ≥99.5%; and a purity of the sodium hydroxide is ≥99.5%, and a purity of the zinc sulfate is ≥99%.

10. An application of a molybdenite/ZnO nanorod-based pillared structure, wherein the pillared structure molybdenite/ZnO nanorod obtained by the preparation method according to any one of claims 1 to 9 is used to monitor ppb-level NO₂ at a room temperature condition.
Correspondence results of Claims of the captioned
application and Allowed Claims of CN application

| Captioned | Allowed CN | |
|---|---|---|
| application | application | Explanation |
| (claims in US | (Allowable claims | Regarding |
| application) | in OEE Application) | the Correspondence |
| 1 | 1 | Independent claim, same |
| | | or similar scope |
| 2 | 2 | Same or similar scope |
| 3 | 3 | Same or similar scope |
| 4 | 4 | Same or similar scope |
| 5 | 5 | Same or similar scope |
| 6 | 6 | Same or similar scope |
| 7 | 7 | Same or similar scope |
| 8 | 8 | Same or similar scope |
| 9 | 9 | Sub-claim of multiple |
| | | dependent claim, same |
| | | or similar scope |
| 10 | 10 | Sub-claim of multiple |
| | | dependent claim, same |
| | | or similar scope |
| 11 | 9 | Sub-claim of multiple |
| | | dependent claim, same |
| | | or similar scope |
| 12 | 9 | Sub-claim of multiple |
| | | dependent claim, same |
| | | or similar scope |
| 13 | 9 | Sub-claim of multiple |
| | | dependent claim, same |
| | | or similar scope |
| 14 | 9 | Sub-claim of multiple |
| | | dependent claim, same |
| | | or similar scope |
| 15 | 9 | Sub-claim of multiple |
| | | dependent claim, same |
| | | or similar scope |
| 16 | 9 | Sub-claim of multiple |
| | | dependent claim, same |
| | | or similar scope |
| 17 | 9 | Sub-claim of multiple |
| | | dependent claim, same |
| | | or similar scope |
